(19) United States
(12) Patent Application Publication
(10) Pub. No.: US 2011/0155130 A1
(43) Pub. Date: Jun. 30, 2011

(54) Dronabinol Treatment for Migraines

(75) Inventor: Lou Barbato, Marietta, GA (US)

(73) Assignee: Unimed Pharmaceuticals, Inc., Marietta, GA (US)

(21) Appl. No.: 13/045,275

(22) Filed: Mar. 10, 2011

Related U.S. Application Data
(63) Continuation of application No. 11/471,891, filed on Jun. 20, 2006, now abandoned.
(60) Provisional application No. 60/691,788, filed on Jun. 20, 2005.

(51) Int. Cl. A61K 31/352; A61P 25/06; A61M 15/00

(52) U.S. Cl. 128/203.12; 514/454

(57) ABSTRACT

In various embodiments, the present invention provides pharmaceutical compositions comprising delta-9-tetrahydrocannabinol and methods of administering such compositions to treat migraines.

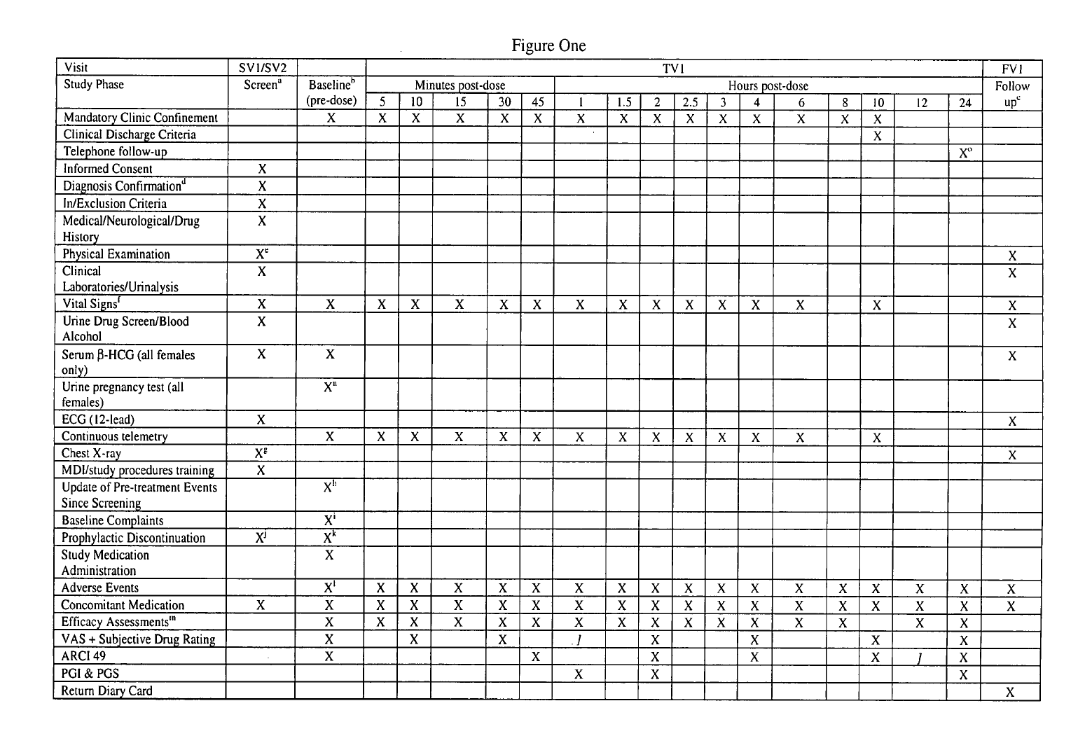

### CROSS REFERENCES TO RELATED APPLICATIONS

This application is a continuation of prior U.S. patent application Ser. No. 11/471,891, filed Jun. 20, 2006, which claims priority to U.S. Provisional Application Ser. No. 60/691,788 filed Jun. 20, 2005 the entire contents of which is hereby incorporated by reference herein.

### FIELD OF THE INVENTION

The present invention relates to the use of pharmaceutical compositions comprising delta-9-tetrahydrocannabinol (“delta-9-THC” or “THC”) as a treatment for migraines.

### BACKGROUND OF THE INVENTION

The marijuana plant has a long history of use for its medicinal properties. The beneficial medical effects of marijuana are mostly attributed to cannabinoids, which are unique to the cannabis plant. Delta-9-THC is the major active cannabinoid responsible for the psychoactive properties. Some of the reported therapeutic effects of THC include being an analgesic, anti-spasmodic, anti-convulsant, anti-tremor, anti-psychotic, anti-inflammatory, anti-emetic, and appetite-stimulant.

Two selective cannabinoid receptor subtypes, CB1 and CB2, have been identified. The CB1 receptors are widely distributed in the central nervous system (“CNS”), especially the limbic system and basal ganglia circuits of the brainstem region. Although less abundant, the CB1 receptors are also located in the peripheral nervous system, reproductive system, immune cells, and gastrointestinal system. The CB2 receptors in the CNS are only present in microglia, mast cells, and the CNS cells associated with anti-inflammatory and immunosuppressive responses.

Migraines should be treated with prompt administration of analgesics at the onset of attack. First line drugs used for the acute treatment of migraine attack include nonspecific and migraine-specific agents. Non-specific drug products, such as aspirin, acetaminophen, nonsteroidal anti-inflammatory agents (“NSAIDs”), opiates, and combination analgesics, are used to treat a wide range of pain disorders. Migraine-specific drug products include ergotamine, dihydroergotamine, and the triptans. Ergotamine-containing products were once the mainstay of therapy, but have been largely replaced by the triptans (e.g., sumatriptan). The triptans, serotonin 1B/1D receptor agonists, work in part by constricting the painfully dilated cerebral blood vessels, but carry the potential to cause coronary vasospasm.

Despite the introduction of the triptans, migraine sufferers still experience inadequate efficacy and frequent troubling side effects, particularly those involved with the cardiovascular system. In addition, the use of triptans in subjects with coronary artery disease is contraindicated. Patients treated with ergot derivatives often experience erratic and potent vasoconstrictor effects, which are associated with adverse vascular events (e.g., stroke, myocardial infarctions), as well as high risk of overuse syndromes and rebound headaches. Other challenges in the therapy for migraine headaches include variability of the response among groups of individuals prescribed the same agent, and from headache to headache within the same individual, as well as variable efficacy among therapeutic agents for different clinical manifestation of migraine headaches, such as absence or presence of aura, duration of headache, severity, and intensity of the headache. Thus, there is a need for an agent, which affects a different brain receptor system (e.g., the CB1 receptor) and offers a better alternative to existing drug products for the acute treatment of migraine headaches.

### SUMMARY OF THE INVENTION

In one embodiment, the present invention provides pharmaceutical compositions comprising delta-9-THC and to methods of administering such compositions to a patient in need of delta-9-THC therapy.

In another embodiment, the present invention provides pharmaceutical compositions comprising delta-9-THC and methods of administering such compositions to treat migraines.

In still another embodiment, acute migraine headaches may be treated by administering a CB1 receptor agonist. Such an agonist can be a member of the cannabinoid family of compounds, such as, for example, delta-9-tetrahydrocannabinol also know as dronabinol.

In yet another embodiment, acute migraine headaches may be treated by administering dronabinol via a pulmonary route, such as through the use of inhalation technologies like metered dose inhalers or nebulizers.

### BRIEF DESCRIPTION OF THE DRAWINGS

FIG. One is an overall schedule of assessments.

### DETAILED DESCRIPTION OF THE INVENTION

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any way. Embodiments illustrated under any heading may be combined with embodiments illustrated under any other heading.

The use of numerical values in the various ranges specified in this application, unless expressly indicated otherwise, are stated as approximations as though the minimum and maximum values within the stated ranges were both preceded by the word “about.” In this manner, slight variations above and below the stated ranges can be used to achieve substantially the same results as values within the ranges. As used herein, the terms “about” and “approximately” when referring to a numerical value shall have their plain and ordinary meanings to one skilled in the art of pharmaceutical sciences or the art relevant to the range or element at issue. The amount of broadening from the strict numerical boundary depends upon many factors. For example, some of the factors to be considered may include the criticality of the element and/or the effect a given amount of variation will have on the performance of the claimed subject matter, as well as other considerations known to those of skill in the art. Thus, as a general matter, “about” or “approximately” broaden the numerical value. For example, in some cases, “about” or “approximately” may mean ±5%, or ±10%, or ±20%, or ±30% depending on the relevant technology. Also, the disclosure of ranges is intended as a continuous range including every value between the minimum and maximum values recited.

It is to be understood that any ranges, ratios, and ranges of ratios that can be formed by any of the numbers or data present herein represent further embodiments of the present invention. This includes ranges that can be formed that do or do not include a finite upper and/or lower boundary. Accordingly, the skilled person will appreciate that such ratios, ranges, and values are unambiguously derivable from the data presented herein.

As used herein, the term “prevent” shall have its plain and ordinary meaning to one skilled in the art of pharmaceutical or medical sciences. Moreover, “prevent” shall mean to stop or hinder a migraine headache.

As used herein, the term “reduce” shall have its plain and ordinary meaning to one skilled in the art of pharmaceutical or medical sciences. In addition, “reduce” shall mean to diminish or decrease the number of occurrences, the duration, or the intensity, of a migraine headache.

As used herein, the terms “treat” and “treating” shall have their plain and ordinary meaning to one skilled in the art of pharmaceutical or medical sciences. Further, “treat” and “treating” shall mean to prevent or reduce a migraine headache.

As used herein, the terms “delta-9-THC” or “THC” are understood to refer to both natural and synthetic delta-9-tetrahydrocannabinol (e.g., dronabinol), and includes all salts, isomers, enantiomers, esters, prodrugs, and derivatives of delta-9-THC.

Anecdotal reports have indicated the potential of marijuana in relieving migraine headaches. Although there are no conclusive clinical data or published surveys about the effect of cannabinoids on migraines, there is a possible link between cannabinoids and migraines as suggested by the abundance of cannabinoid receptors in the periaqueductal gray (“PAG”) region of the brain. The PAG region is part of the neural system that suppresses pain and is thought to be involved in the generation of migraine headaches. Evidence of PAG involvement in cannabinoid-induced antinociception had been demonstrated in rats.

In addition, a systematic review of randomized controlled clinical trials indicated that 5 mg to 20 mg oral doses of THC were as effective as 50 mg to 120 mg of codeine. Butorphanol tartrate (e.g., Stadol®), an opiate agonist/antagonist, has been shown to be beneficial for acute migraine attacks with onset of pain relief within 15 minutes. Studies have indicated both pharmacological and biochemical interactions between opioids and cannabinoids. Thus, the potential interaction between the endocannabinoid and opiate systems for dronabinol may provide the same benefit as butorphanol tartrate in the acute treatment of migraines.

Among several hypotheses, platelet hyperaggregability and release of platelet serotonin have been implicated in the pathogenesis of migraines. The inflammatory mediators such as bradykinin, histamine, prostaglandins, leukotrienes, etc. released as a result of vasodilatation are potent platelet receptor agonists, which are responsible for activation of platelets to aggregate and to release serotonin (“5HT”). Serotonin is a potent algesic substance which excitatory action on 5HT3 receptors of nociceptors at nerve endings potentiates the algesic effects of the mediators. Cannabinoids have been shown to inhibit platelet aggregation and release of serotonin.

A synthetic version of delta-9-THC, dronabinol, has been developed for medicinal purposes and has been marketed in the U.S. and elsewhere as an oral formulation sold under the trade name, MARINOL®. MARINOL® has been approved for use in the treatment of nausea and vomiting following cancer chemotherapy in the United States since 1985.

Treatment of acute migraine headaches (including migraines with and without associated aura) and associated symptoms, and methods of providing relief of associated symptoms in subjects may be accomplished by administering an amount of a CB1 receptor, such as a cannabinoid, for example such as dronabinol. Such an amount can include, for example, high, medium and/or low dosages. Dosing regimes can be at the onset of initial symptoms, or within two hours after the onset of a migraine attack and may be single dose or multi doses, for example, every two hours, or even every four, six, or eight hours as needed.

in one embodiment, the dose of delta-9-THC received by a patient according to methods of the present invention may be, for example, about 1 to about 50 mg, about 2 mg to about 20 mg, or about 2 mg to about 10 mg per day. For example, a patient according to methods of the present invention may receive about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 16.0, 17.0, 18.0, 19.0, 20.0, 21.0, 22.0, 23.0, 24.0, 25.0, 26.0, 27.0, 28.0, 29.0, 30.0, 31.0, 32.0, 33.0, 34.0, 35.0, 36.0, 37.0, 38.0, 39.0, 40.0, 41.0, 42.0, 43.0, 44.0, 45.0, 46.0, 47.0, 48.0, 49.0 or 50.0 mg of delta-9-THC per day. The doses described herein may be administered once to a small plurality of times per day, for example about 1, 2, 3, 4, 5, or 6 times per day.

In one embodiment, treatment of acute migraine headaches can include a method of treating the intensity of pain associated with acute migraine headaches by administering dronabinol to subjects having a pain intensity rating of greater than 0 such as, for example, a pain intensity rating of 1, 2 or 3.

In another embodiment, a treatment of acute migraine headaches can include a method of providing pain relief, such as pain relief associated with acute migraine headaches, in subjects by administering dronabinol to minimize or eliminate the pain, for example, by achieving or maintaining an effective pain relief rating, such as, for example, a rating of greater than 0, such as 1 or 2, and more preferably 3 or 4.

In yet another embodiment, a treatment of acute migraine headaches can include a method of treating nausea associated with acute migraine headaches in subjects by administering dronabinol to achieve or maintain an effective nausea intensity rating, for example, a nausea intensity rating of less than 2, such as 1, or preferably 0.

In another embodiment, treatment of acute migraine headaches can include a method of treating vomiting associated with acute migraine headaches in subjects by administering dronabinol to achieve or maintain an effective vomiting rating, for example, a vomiting rating of 0.

In another embodiment, treatment of acute migraine headaches can include a method of treating photophobia associated with acute migraine headaches in subjects by administering dronabinol to achieve or maintain an effective photophobia rating, for example, a photophobia rating of 0.

In another embodiment, treatment of acute migraine headaches can include a method of treating phonophobia associated with acute migraine headaches in subjects by administering dronabinol to achieve or maintain an effective phonophobia rating, for example, a phonophobia rating of 0.

In another embodiment, treatment of acute migraine headaches can include a method of treating functional disability associated with acute migraine headaches in subjects by administering dronabinol to achieve or maintain an effective functional disability rating, for example, a functional disability rating less than 3, such as 2, more preferably 1, or even 0.

In another embodiment, treatment of acute migraine headaches can include improving a patient's global impression with treatment of acute migraine headaches by administering dronabinol to achieve or maintain an effective patient global impression of improvement rating, for example, a patient global impression of improvement rating less than 4, such as 3 or 2, more preferably 1.

In another embodiment, treatment of acute migraine headaches can include providing patient global satisfaction with treatment of acute migraine headaches by administering dronabinol to achieve or maintain an effective patient global impression of treatment satisfaction rating, for example, patient global impression of treatment satisfaction rating less than 3, such as 2, or more preferably, 1.

In another embodiment, treatment of acute migraine headaches can include reducing the use of rescue medication in a subject with acute migraine headaches by administering dronabinol to achieve or maintain an effective patient reduction in the use of rescue medication in a subject with acute migraine headaches, for example, a reduction of 1 or more uses, such as a reduction to 1 use or less, such as 0 uses.

In another embodiment, treatment of acute migraine headaches can include decreasing time to onset of meaningful relief in a subject with acute migraine headaches by administering dronabinol to achieve or maintain an effective time to onset of meaningful relief in a subject with acute migraine headaches, for example, a time to onset of meaningful relief of 2 hours or less, such as 1.5 hours, or 1 hour, or even 0.5 hours.

In another embodiment, treatment of acute migraine headaches can include treatment of aura associated with acute migraine headaches in subjects by administering dronabinol to achieve removal of or reduction in patient perceived aura.

In yet another embodiment, treatment of acute migraine headaches can include treatment of the PAG region of the brain in subjects by administering dronabinol in an effective amount.

In still another embodiment, treatment of acute migraine headaches can include reduction of or inhibition of platelet aggregation and release of serotonin by administering dronabinol in an effective amount.

In one embodiment, compositions of the present invention are in the form of an orally deliverable dosage unit. The terms “oral administration” or “orally deliverable” herein include any form of delivery of a therapeutic agent or a composition thereof to a subject wherein the agent or composition is placed in the mouth of the subject, whether or not the agent or composition is swallowed. Thus “oral administration” includes buccal and sublingual as well as esophageal administration.

Compositions of the present invention can be formulated as solid, liquid, or semi-solid dosage forms. In one embodiment, such compositions are in the form of discrete dose units or dosage units. The terms “dose,” “dose unit,” and/or “dosage unit” herein refer to a portion of a pharmaceutical composition that contains an amount of a therapeutic agent suitable for a single administration to provide a therapeutic effect. Such dosage units may be administered one to a small plurality (e.g., 1 to about 4) times per day, or as many times as needed to elicit a therapeutic response. A particular dosage form can be selected to accommodate any desired frequency of administration to achieve a specified daily dose. Typically one dose unit, or a small plurality (e.g., up to about 4) of dose units, provides a sufficient amount of the active drug to result in the desired response or effect.

Alternatively, compositions of the invention can also be formulated for rectal, topical, transdermal, or parenteral (e.g., subcutaneous, intramuscular, intravenous and intradermal or infusion) delivery. In one embodiment, compositions of the invention can be formulated as a patch, gel, lotion, ointment, cream, or spray.

In another embodiment, a single dosage unit, be it solid or liquid, comprises a therapeutically and/or prophylactically effective amount of dronabinol. The term “therapeutically effective amount” or “therapeutically and/or prophylactically effective amount” as used herein refers to an amount of compound or agent that is sufficient to elicit the required or desired therapeutic and/or prophylactic response, as the particular treatment context may require.

It will be understood that a therapeutically and/or prophylactically effective amount of a drug for a patient is dependent inter alia on the body weight of the patient. A “patient” herein to which a therapeutic agent or composition thereof can be administered includes a human subject of either sex and of any age, and also includes any nonhuman animal, particularly a domestic or companion animal, illustratively a cat, dog, or a horse.

In various embodiments, compositions of the invention are in the form of solid dosage forms or dosage units. Non-limiting examples of suitable solid dosage forms include tablets (e.g., suspension tablets, bite suspension tablets, rapid dispersion tablets, chewable tablets, effervescent tablets, bilayer tablets, etc.), caplets, capsules (e.g., a soft or a hard gelatin capsule), powder (e.g., a packaged powder, a dispensable powder, or an effervescent powder), lozenges, sachets, cachets, troches, pellets, granules, microgranules, encapsulated microgranules, powder aerosol formulations, or any other solid dosage form reasonably adapted for oral administration.

In another embodiment, compositions of the invention can be in the form of liquid dosage forms or units. Non-limiting examples of suitable liquid dosage forms include solutions, suspension, elixirs, syrups, liquid aerosol formulations, etc.

In yet another embodiment, compositions of the present invention can be in the form of a metered dose inhaler (“MDI”), such as the metered dose inhaler outlined in co-pending U.S. application Ser. No. 11/361,463, which is incorporated herein by reference. Specifically, the present invention can be in the form of a metered dose inhaler comprising about 0.5% delta-9-THC, about 10% ethanol (dehydrated alcohol), and about 89.5% Propellant HFA-134a (1,1,1,2 tetrafluoroethane). In another embodiment, the present invention can be in the form of a metered dose inhaler comprising about 2.0% delta-9-THC, about 10% ethanol (dehydrated alcohol), and about 88.0% Propellant HFA-134a (1,1,1,2 tetrafluoroethane).

Compositions of the invention optionally comprise one or more additional pharmaceutically acceptable excipients. The term “excipient” herein means any substance, not itself a therapeutic agent, used as a carrier or vehicle for delivery of a therapeutic agent to a subject or added to a pharmaceutical composition to improve its handling or storage properties or to permit or facilitate formation of a unit dose of the composition.

Illustrative excipients include antioxidants, surfactants, adhesives, agents to adjust the pH and osmolarity, preservatives, thickening agents, colorants, buffering agents, bacteriostats, stabilizers, and penetration enhancers. Generally speaking, a given excipient, if present, will be present in an amount of about 0.001% to about 95%, about 0.01% to about 80%, about 0.02% to about 25%, or about 0.3% to about 10%, by weight.

Illustrative antioxidants for use in the present invention include, but are not limited to, butylated hydroxytoluene, butylated hydroxyanisole, potassium metabisulfite, and the like. One or more antioxidants, if desired, are typically present in a composition of the invention in an amount of about 0.01% to about 2.5%, for example about 0.01%, about 0.05%, about 0.1%, about 0.5%, about 1%, about 1.5%, about 1.75%, about 2%, about 2.25%, or about 2.5%, by weight.

In various embodiments, compositions of the invention comprise a preservative. Suitable preservatives include, but are not limited to, benzalkonium chloride, methyl, ethyl, propyl or butylparaben, benzyl alcohol, phenylethyl alcohol, benzethonium, or combination thereof. Typically, the optional preservative is present in an amount of about 0.01% to about 0.5% or about 0.01% to about 2.5%, by weight.

In one embodiment, compositions of the invention optionally comprise a buffering agent. Buffering agents include agents that reduce pH changes. Illustrative classes of buffering agents for use in various embodiments of the present invention comprise a salt of a Group IA metal including, for example, a bicarbonate salt of a Group IA metal, a carbonate salt of a Group IA metal, an alkaline or alkali earth metal buffering agent, an aluminum buffering agent, a calcium buffering agent, a sodium buffering agent, or a magnesium buffering agent. Suitable buffering agents include carbonates, phosphates, bicarbonates, citrates, borates, acetates, phthalates, tartrates, succinates of any of the foregoing, for example sodium or potassium phosphate, citrate, borate, acetate, bicarbonate and carbonate.

Non-limiting examples of suitable buffering agents include aluminum, magnesium hydroxide, aluminum glycinate, calcium acetate, calcium bicarbonate, calcium borate, calcium carbonate, calcium citrate, calcium gluconate, calcium glycerophosphate, calcium hydroxide, calcium lactate, calcium phthalate, calcium phosphate, calcium succinate, calcium tartrate, dibasic sodium phosphate, dipotassium hydrogen phosphate, dipotassium phosphate, disodium hydrogen phosphate, disodium succinate, dry aluminum hydroxide gel, magnesium acetate, magnesium aluminate, magnesium borate, magnesium bicarbonate, magnesium carbonate; magnesium citrate, magnesium gluconate, magnesium hydroxide, magnesium lactate, magnesium metasilicate aluminate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, sodium bicarbonate, sodium borate, sodium carbonate, sodium citrate, sodium gluconate, sodium hydrogen phosphate, sodium hydroxide, sodium lactate, sodium phthalate, sodium phosphate, sodium polyphosphate, sodium pyrophosphate, sodium sesquicarbonate, sodium succinate, sodium tartrate, sodium tripolyphosphate, synthetic hydrotalcite, tetrapotassium pyrophosphate, tetrasodium pyrophosphate, tripotassium phosphate, trisodium phosphate, and trometarnol. (Based in part upon the list provided in The Merck Index, Merck & Co. Rahway, N.J. (2001)). Furthermore, combinations or mixtures of any two or more of the above mentioned buffering agents can be used in the pharmaceutical compositions described herein. One or more buffering agents, if desired, are present in compositions of the invention in an amount of about 0.01% to about 5% or about 0.01% to about 3%, by weight.

The foregoing excipients can have multiple roles as is known in the art. For example, some flavoring agents can serve as sweeteners as well as a flavoring agent. Therefore, the classification of the excipients above is not to be construed as limiting in any manner.

These and many other aspects of the invention will be fully apparent to one of ordinary skill in the art in view of the example set forth below. The example provided herein is illustrative and one that the applicant will perform. Therefore, the example is not to be construed as limiting the invention in any manner.

### Example

### Study Objectives

The primary objective of this study will be to evaluate the efficacy, safety and tolerability of dronabinol MDI versus placebo for the acute treatment of a single moderate to severe migraine headache attack. The primary efficacy parameter will be defined as the proportion of subjects who experience pain response (pain intensity score of mild=1 or none=0) at two hours post-dose without the use of any rescue medication.

Secondary objectives will be to determine the potential effective dose(s) of dronabinol MDI for the acute treatment of migraine headaches and to assess the single dose subjective effects and preliminary abuse liability of dronabinol MDI in subjects with migraine headaches.

### Safety

Safety assessments will include a medical, neurological and drug history, physical examination, clinical hematology and biochemistry assessments, urine and alcohol drug screens, urinalysis, chest X-ray, vital signs (pulse rate, blood pressure and temperature), continuous telemetry monitoring during confinement, electrocardiogram (“ECG”), concomitant medications and adverse event monitoring. In addition, the single dose subjective effects and preliminary abuse liability of dronabinol MDI will also be evaluated.

### Study Design

This study will be designed as a multicenter, randomized, double-blind, placebo-controlled, efficacy, safety and tolerability study for the acute treatment of migraine headaches with or without aura. Subjects who consent to participate in the study and meet the inclusion/exclusion criteria at the screening visit(s) (either SV1 or SV2) will be qualified.

Eligible subjects will be requested to report to the clinic within 2 hours after the onset of a migraine attack for the treatment visit (“TV1”), which includes a minimum ten-hour mandatory observation period post-dose. A total of 240 eligible subjects who present at the clinic (TV1) with a moderate to severe migraine headache and meet the treatment criteria will be randomized in a 1:1:1:1 ratio to receive high (3.6 mg) or medium (2.4 mg) or low (1.2 mg) dose of dronabinol MDI or placebo. Systemic delivery of dronabinol via the lungs will be accomplished by using a pressurized MDI. Subjects will be instructed to self-administer a dose of the study medication at the clinic with close observation up to two hours post-dosing under the direct supervision of the site Investigator or Sub-Investigator for treatment of a moderate to severe migraine headache attack. The headache pain intensity will be defined using a four-point scale of no pain or none (0), mild (1), moderate (2) and severe (3). In addition, medically qualified and trained site personnel will closely monitor subjects from 2 hours up to a minimum of ten hours post-dose.

Rescue medication will be allowed at two or more hours post administration of study medication. Medically stable subjects will be eligible for release from the clinic no sooner than ten hours post treatment at the discretion of the Investigator in accordance with the pre-defined clinic discharge criteria. The discharge criteria checklist will require the Investigator or Sub-Investigator to ensure that the cardiovascular parameters are not in the markedly abnormal range, any ECG changes have returned to normal, any serious adverse events (“SAE”) have been treated appropriately, and all adverse events (“AE”) and indicators of psychotropic effects are appropriately assessed prior to release. Released subjects will be instructed to return to the clinic 24 to 72 hours after dosing for a follow-up visit (“FV1”).

Safety assessments will include medical, neurological and drug history, physical examination, clinical hematology and biochemistry, urine and alcohol drug screen, urinalysis, chest X-ray, vital signs (pulse rate, blood pressure and temperature), ECG, continuous telemetry monitoring during confinement, adverse events monitoring and concomitant medication use at screening, baseline, during drug treatment and/or at follow-up visit.

During clinic confinement, subjects will be closely monitored for ECG changes or marked vital sign changes through continuous telemetry with real-time evaluation by a centralized cardiologist at 0 (pre-dose, baseline), 5, 10, 15, 30, 45 minutes and at 1, 1.5, 2, 2.5, 3, 4, 6 and 10 hours post-dosing.

Efficacy and subjective effect assessments for the treated attack will be self-rated using subject diaries before dosing (baseline) and periodically at specific time points up to 24 hours post-dose. In addition, adverse events, cardiovascular and psychotropic safety parameters will be closely monitored and recorded. The use of concomitant medications will also be recorded in subject diaries. Follow-up assessments will be completed at the clinic during FV1. The completed diaries will be collected by the Investigator at FV1.

Eligible subjects who fail to return to the clinic for a treatment visit (TV1) within, six weeks after the screening visit(s) (either SV1 or SV2) will be contacted by the Investigator for repeat procedural training. Subjects who fail to enter the study within three months after qualifying for treatment will be terminated from the study and may not re-enter.

### Subject Selection

### Study Assessments and Conduct

The overall schedule of assessments is summarized in FIG. One. The following list provides specific information in FIG. One's Schedule of Assessments: a.) the screening visit (SV1) can last up to 14 days to allow for repeat of abnormal laboratory or other tests (SV2); b.) subjects will be randomized to receive study medication at the treatment visit (TV1) only after treatment criteria have been confirmed; c.) end-of-study follow-up visit (FV1) in 24 to 72 hours after dosing; d.) diagnostic assessments based on IHS Criteria for Migraine with or without aura; e.) physical examination to include height (at screening only) and body weight; f.) vital signs include supine pulse rate and blood pressure, and temperature (at screening and follow-up only); g.) performed at screening visit or within 14 days prior to screening; h.) pre-baseline events assessment may be performed anytime during TV1—number of attacks experienced during period from screening to treated attack will also be collected; i.) assessment to be performed prior to study medication; j.) migraine prophylactic medications are allowed until onset of headache for the treated attack; k.) confirmation of migraine prophylactic discontinuation; l.) assessment to be performed post study medication; m.) assessment to be performed during waking hours; n.) urine pregnancy test at baseline (pre-dose) must be negative prior to randomization and dosing; and o.) contact by clinic staff for subjects who failed to return to the clinic within 24 hours of release.

### Efficacy Assessments

Efficacy of the study medication will be self-evaluated by study subjects using diary cards. Measures of efficacy include: 1.) pain intensity; 2.) use of rescue medication; 3.) pain relief; 4.) time to onset of meaningful relief; 5.) functional disability; 6.) nausea intensity; 7.) vomiting; 8.) photophobia; 9.) phonophobia; 10.) patient global impression of improvement (“PGI”); and 11.) patient global impression of treatment satisfaction (“PGS”).

All efficacy measures, except time to meaningful relief, PGI and PGS, are assessed at 0 (pre-dose, baseline), 5, 10, 15, 30 and 45 minutes, at 1, 1.5, 2, 2.5, 3, 4, 6, 8, 12 and 24 hours post-dose (while awake).

Pain intensity will be rated on a scale of 0=no pain, 1=mild pain allowing normal activity, 2=moderate pain, which is disturbing but does not prohibit normal activity and require bed rest, and 3=severe pain, which prohibits normal activity and requires bed rest.

Pain relief as compared to baseline will be rated as 0=no relief, 1=slight relief, 2=moderate relief, 3=lots of relief, and 4=complete relief. Nausea intensity will be rated as 0=no nausea, 1=mild nausea, 2=moderate nausea, and 3=severe nausea. Vomiting, photophobia and phonophobia will be rated as 0=absent and 1=present.

Functional disability will be rated on a scale of 0=no disability: able to function normally, 1=performance of daily activities mildly impaired: can still do everything but with difficulties, 2=performance of daily activities moderately impaired: unable to do some things and 3=performance of daily activities severely impaired: cannot do all or most things, bed rest may be necessary.

The time to obtain meaningful pain relief will be defined subjectively by the subjects using the stopwatch method. Subjects will be instructed to indicate on the diary cards the relative time after dosing when they feel they have reached “meaningful relief.” The assessment period will be for the first two hours after administration of study medication.

PGI will be assessed at 1, 2, and 24 hours post-dose as a change from onset of the headache to evaluate the overall experience and expectation with treatment. PGI assessment will be rated as 1=very much improved, 2=much improved, 3=minimally improved, 4=no change, 5=minimally worse, 6=much worse, and 7=very much worse.

PGS will be assessed at 1, 2, and 24 hours post-dose as a general rating score of satisfaction with the treatment or the preference to using it again. PGS will be rated as 1=very satisfied, 2=somewhat satisfied, 3=neither satisfied nor dissatisfied, 4=somewhat dissatisfied, and 5=very dissatisfied.

If rescue medication is necessary at two or more hours post-dose during the 24-hour evaluation period, subjects will also be instructed to record each time and the respective name of the rescue medication used.

### Abuse Liability Assessments

Eight visual analog scales (“VAS”) with increment values ranging from 0 (no effect/not at all) to 100 (maximum/very much) will be used to evaluate the subjective effect of dronabinol MDI. Subjects will be asked the following questions: (1) How much of a drug effect do you feel?; (2) How much do you like the drug?; (3) How much do you dislike the drug?; (4) How high do you feel?; (5) Do you want to take more of the drug?; (6) Do you feel stimulated?; (7) Do you feel anxious?; and (8) Do you feel sedated?. These assessments will be conducted at 0 (pre-dose, baseline), 10, 30 minutes and 2, 4, 10, and 24 hours post-dose.

The Addiction Research Center Inventory (“ARCI”) short form will also be used to evaluate the psychoactive effect of dronabinol MDI. The ARCI is a “true-false” questionnaire developed specifically to measure drug-induced euphoria, sedation and dysphoria. These assessments will be conducted at 0 (pre-dose, baseline), 45 minutes and at 2, 4, 10, and 24 hours post-dose.

The ARCI will be completed after the VAS when both assessments are performed at the same time point. Verbal assistance from clinic staff on these assessments will be allowed.

### Safety Assessments

The following safety assessments will be included in this study: (1) medical, neurological and drug history; (2) physical examination to include height (at screening only) and body weight; (3) clinical laboratory assessments (hematology, biochemistry, and urinalysis; urine and β-HCG for all females); (4) urine drug and blood alcohol screens; (5) chest X-ray; (6) vital signs to include pulse rate and blood pressure after five minutes rest in supine position and temperature; (7) electrocardiogram (12-lead ECG) at screening and follow-up, and continuous telemetry at baseline and for ten hours after dosing during confinement; (8) adverse event monitoring throughout treatment; (9) pre-baseline event assessment since the screening visit; (10) baseline complaints; (11) concomitant medication use; and (12) update of events from screening to baseline.

Medical, neurological and drug history, physical examination, 12-lead ECG, chest X-ray, clinical laboratory assessments to include β-HCG for females only (screening and baseline), drug and alcohol screens, and vital signs will be performed at the initial screening visit (SV1). Repeat of abnormal laboratory (or other) tests will be performed at the second screening visit (SV2) as necessary. Adverse events, including baseline and ten hours post-treatment continuous telemetry, will be monitored after study drug administration and for the remainder of the study. Chest X-ray, 12-lead ECG, physical examination, vital signs, clinical laboratory assessments, drug and alcohol screens and β-HCG for females only will be performed at the follow-up visit.

The Investigator or Sub-Investigator will be present at dosing and for up to two hours following dosing to monitor subject safety. Medically qualified and trained site personnel (e.g., Investigator, Sub-Investigator or study coordinator) will closely monitor subjects after two hours and for up to a minimum of ten hours after dosing. Subjects must be medically stable in accordance with the pre-defined clinic discharge criteria at ten hours after dosing to be eligible for release from the clinic.

Following release from the clinic, subjects will be provided with a 24-hour hotline telephone number in the event medical assistance is needed prior to the scheduled follow-up visit. Site personnel will telephone subjects who have not returned to the clinic within 24 hours after discharge and remind them of the scheduled follow-up visit (due within 72 hours of release).

Pre-arranged transportation will be provided on a 24-hour basis to all subjects to and from the clinic for dosing, and to and from the clinic for post-dosing evaluation. In addition, an identification card intended for employers and law enforcement personnel will be provided to all subjects upon request confirming their participation in the study.

### Medical History and Physical Examination

A complete medical and neurological history and physical examination are to be performed for each subject at the screening visit to determine eligibility for the clinical study. Medical history and physical examinations to be assessed include the following as listed in Table 1 and Table 2, respectively.

TABLE 1
Medical History Assessments

| | |
|---|---|
| Blood and lymphatic system | Nervous system disorders |
| disorders | Pregnancy, puerperium and |
| Cardiac disorders | perinatal conditions |
| Congenital and familial/genetic | Psychiatric disorders |
| disorders | Renal and urinary disorders |
| Ear and labyrinth disorders | Reproductive system and breast |
| Endocrine disorders | disorders (incl. sexual functioning) |
| Gastrointestinal disorders | Respiratory, thoracic and |
| General disorders and administration | mediastinal disorders |
| site conditions | Skin and subcutaneous tissue |
| Hepato-biliary disorders | disorders |
| Immune system disorders | Social circumstances |
| Infections and infestations | Special senses (vision, hearing, |
| Injury and poisoning | taste, smell, touch) |
| Investigations | Surgical and medical procedures |
| Metabolism and nutrition disorders | Vascular disorders |
| Musculoskeletal, connective tissue | Allergies (drug, non-drug) |
| and bone disorders | Recreational drug use |
| Neoplasms benign and malignant | Drug abuse, alcohol and smoking |
| (including cysts and polyps) | habits |
| | Previous and/or current |
| | medication/therapy |

TABLE 2
Physical Examination Assessments

| | |
|---|---|
| Head, ears, eyes, nose, throat | Abdomen |
| Cardiovascular | Musculoskeletal |
| Respiratory | Gross neurological |
| Major lymph nodes | Skin |
| | Other |

### Vital Signs

Vital signs (blood pressure, pulse rate and temperature) will be measured while the subjects are at rest in supine position for at least five minutes. The subject's temperature will only be taken at screening and follow-up.

### Electrocardiogram/Continuous Telemetry

Standard 12-lead ECGs will be recorded after five minutes of rest in supine position using an automatic device. The different ECG intervals (PQ, QRS, QT) and HR will be determined. The QT-interval will be corrected for heart rate.

All ECGs will be transmitted to a centralized laboratory to be reviewed by a qualified cardiologist. Continuous telemetry during the 10-hour confinement period will include real-time evaluation by the cardiologist at the centralized laboratory with feedback readily available back to the Investigator site within two hours.

In addition, all ECGs will be reviewed periodically by a board certified cardiologist at the centralized laboratory for data trend. Data including standard reports, data displays, graphs and charts, as well as the actual annotated ECG tracing will be available via a secure Web portal.

### Pharmacokinetic Assessments

No pharmacokinetic assessments are planned for this study.

### Laboratory Assessments

Laboratory assessments will be performed at screening and follow-up visit. All laboratory samples will be processed via a centralized laboratory. A list of laboratory assessments is provided in Table 3. The Investigator will note the review of the laboratory results by initialing and dating the report provided by the central laboratory in a timely fashion. Out of range values will be interpreted by the Investigator with a comment of not clinically significant (“NCS”) or clinically significant (“CS”) with a comment to planned follow-up. Clinically significant abnormal laboratory values must be repeated, or the clinical follow-up arranged by the clinical Investigator and documented on the laboratory report, until stabilized or they have returned to within the acceptable range regardless of the relationship to study medication therapy. Any clinically significant abnormality ongoing at termination will be followed according to accepted medical standards for up to 30 days or until resolution of the abnormality.

TABLE 3
Laboratory Assessments

| | Blood | | | Urine Drug |
|---|---|---|---|---|
| Hematology | Chemistry | Special tests | Urinalysis | Screen* |
| Screening and | Screening and | Screening and | Screening | Screening and |
| follow-up | follow-up | follow-up | and follow- | follow-up |
| visit | visit | visit | up visit | visit** |
| Hemoglobin | Sodium | Blood alcohol | pH | Barbiturates |
| Hematocrit | Potassium | Screening, | Specific | Benzodiazepines |
| WBC w/ | Chloride | treatment and | Gravity | Cannabinoids |
| differential | Bicarbonate | follow-up | Glucose | Cocaine |
| RBC | Calcium | visit | Protein | Methadone |
| MCV | Urea nitrogen | Pregnancy | Microscopic | Methaqualone |
| MCH | (BUN) | Test (serum β- | examination | Opiates |
| MCHC | Glucose | HCG) for all | | Phencyclidine |
| Platelet count | Creatinine | females*** | | Amphetamines |
| | Alkaline | Pregnancy | | Propoxyphene |
| | phosphatase | Test (Urine β- | | |
| | Total bilirubin | HCG) for all | | |
| | SGPT/ALT | females at | | |
| | SGOT/AST | treatment | | |
| | LDH | visit*** | | |
| | GGT | | | |
| | Triglycerides | | | |
| | Cholesterol | | | |
| | Total protein | | | |
| | Uric acid | | | |

*The subject should be excluded if results are positive at the screening visit. Exceptions will be for documented medications used as acute treatment or prophylaxis for migraine headache.
**Subjects who are randomized to receive active drug treatment or use rescue medications during the study may produce positive results at follow-up visit.
***The subject should be excluded if results are positive at the screening visit. A second β-HCG test will be done at the treatment visit; results will not be available prior to dosing but will not preclude dosing. A urine pregnancy test will also be performed at the treatment visit. The results of the urine pregnancy test must be negative prior to randomization and dosing.

### Screening Visits (SV1/SV2)

The screening period can last up to 14 days to allow for repeat of abnormal laboratory or other tests. The first screening visit is denoted as SV1. The second screening visit for repeat laboratory assessment is denoted as SV2. The screening interview will include: (1) informed consent; (2) confirmation of migraine diagnosis; (3) review of inclusion/exclusion criteria; (4) complete medical, neurological and drug history including history of marijuana use; (5) physical examination including vital signs (supine pulse rate and blood pressure, and temperature), height and body weight; (6) 2-lead ECG; (7) chest X-ray (assessment performed within 14 days prior to screening is acceptable); (8) clinical laboratory assessments including serum pregnancy test for all females, blood alcohol and urine drug screens (see Table 3 for specific tests); and (9) prior and concomitant medications within past three months; the dose regimen of prior and current medications should be reported in addition to therapeutic outcome.

At the screening visit, the diagnosis of migraine will be confirmed by one of the following types of documentation: (1) the Investigator's medical record; (2) a copy of the medical record from the subject's private physician; (3) a letter from the subject's private physician; (4) a notation of a telephone call from the subject's private physician; (5) a letter from a qualified specialist which affirms that the subject's diagnosis of migraine conformed to the IHS criteria; or (6) newly diagnosed subjects during screening visit at the study site with sufficient documents to support a minimum history of one year.

Subjects who qualify for study participation at the screening visit will be trained on the use of the MDI along with efficacy assessment diaries and abuse liability assessments verbatim and a checklist of study procedures.

Treatment Visit (TV1) within Three Months after Qualification

Treatment of a moderate to severe migraine attack should be completed within three months after qualification for participation into the study.

Eligible subjects will be requested to report to the clinic site within two hours after the onset of a migraine attack. Subjects will be randomized and instructed to self-administer actuations as defined in Table 4, at the onset of a moderate to severe migraine attack. Dose administration will be under the direct supervision of the Investigator or Sub-Investigator, who will continue to directly observe the subject for up to two hours after dosing.

Subjects will be required to complete the self-rated efficacy and abuse liability assessments diaries for the treated attack. In addition, subjects will also be requested to complete a checklist of study procedures along with documentation of concomitant medications taken within two days prior to the treated attack. An update of events from the screening visit to baseline will be completed during the clinic visit.

Subjects will be allowed to take rescue medication at two or more hours post administration of study medication if medically necessary. The time and name of rescue medication used each time during the 24-hour treatment period will be recorded. In addition, adverse events will also be recorded in the diaries during the 24-hour study treatment period.

### Follow-Up Visit (FV1)

Each subject is to return to the clinic 24 to 72 hours after dosing. A telephone follow-up will be made by clinic staff to subjects who have not returned to the clinic within 24 hours after release and to remind them of the follow-up visit to be completed within the next 48 hours. Should any adverse event be identified at this visit, the Investigator will continue to follow the subject.

Safety assessments to be performed include chest X-ray, 12-lead ECG, vital signs, physical examination, adverse events monitoring, concomitant medications, clinical laboratory assessments to include β-HCG for females only, urine drug and blood alcohol screens. The completed subject diaries and checklist will be collected.

### Study Medication

### Drug Supplies

Solvay Pharmaceuticals, Inc. will supply sufficient amounts of dronabinol MDI and placebo MDI in this study. The active compound in dronabinol is THC. The chemical name is (6a R-trans)-6a,7,8,10a-tetrahydro-6,6,9-trimethyl-3-pentyl-6H-dibenzo[b,d]pyran-1-ol.

In order to preserve the blind, subjects will self-administer pulmonary doses from each of three MDIs (double-dummy design). Subjects who are randomized to receive placebo will be administered the exact number of actuations as those who receive the active study medication. In addition, all subjects will receive the same number of inhalations from the same number of MDIs, either active and/or placebo, to protect the blinding procedures. The number of actuations to be administered from each MDI for each treatment group is provided in Table 4.

TABLE 4
Number of Actuations per MDI

| Treatment | | | |
|---|---|---|---|
| Group | MDI No. 1 | MDI No. 2 | MDI No. 3 |
| 3.6 mg | 1 per dronabinol | 1 per dronabinol | 1 per dronabinol |
| | MDI | MDI | MDI |
| 2.4 mg | 1 per dronabinol | 1 per dronabinol | 1 per placebo MDI |
| | MDI | MDI | |
| 1.2 mg | 1 per dronabinol | 1 per placebo MDI | 1 per placebo MDI |
| | MDI | | |
| Placebo | 1 per placebo | 1 per placebo MDI | 1 per placebo MDI |
| | MDI | | |

### Dronabinol MDI

The dronabinol MDI to be used in this study is manufactured to deliver 1.2 mg dronabinol per actuation per 50 μl. The MDI consists of a 1.0 ml pressurized (via propellants) container and a 50 μl metered-dose valve. The propellant and solvent employed are 1,1,1,2 tetrafluoroethane 134a (HFA 134a) and ethanol, respectively. Each MDI is capable of delivering 100 actuations. The composition of dronabinol MDI is provided in Table 5. The unit is placed within a mouthpiece (oral adapter, or “actuator”), and upon actuation, an exact amount of drug is expelled in the proper particle size distribution.

TABLE 5
Composition of Dronabinol MDI

| | | Quantity (% w/w) | Quantity (mg) per |
|---|---|---|---|
| Component | Grade | per 10 ml canister | 50 μl actuation |
| Δ⁹-THC | USP | 2.0 | 1.2 |
| Ethanol | USP | 10 | 6.0 |
| Propellant HFA | Pharma | 88.0 | 52.8 |
| 134a | | | |

### Placebo MDI

To maintain the blind, matching placebo MDI will be used. The placebo MDI is capable of delivering 100 actuations as well. The composition of placebo MDI is provided in Table 6.

TABLE 6
Composition of Placebo MDI

| | | Quantity (% w/w) | Quantity (mg) per |
|---|---|---|---|
| Component | Grade | per 10 ml canister | 50 μl actuation |
| Δ⁹-THC | USA | 0 | 0.0 |
| Ethanol | USP | 10 | 6.0 |
| Propellant HFA | Pharma | 90 | 54.0 |
| 134a | | | |

### Dosing Information

Each subject will be randomized and instructed to self-administer one pulmonary dose (corresponding to three actuations) of dronabinol MDI and/or placebo MDI (depending on the treatment group, as defined in Table 4) within two hours after the onset of a moderate to severe migraine attack. All dosing will be under the direct supervision of the Investigator or Sub-Investigator and confined to the clinic.

Rescue medication will be allowed at two or more hours post administration of study medication if medically necessary. Subjects will use rescue medications prescribed by the Investigator. The choice of rescue medication at the discretion of the Investigator should be cautioned against those, specifically the class of triptans, with a potential to cause clinically significant cardiovascular side effects. No rescue medication will be provided by Solvay Pharmaceuticals, Inc.

### Compliance

Study medication will be self-administered by study subjects under supervision of the Investigator or Sub-investigator. Treatment compliance will be evaluated by documentation of study drug used recorded in the subjects' diaries. In addition, MDI will be weighed by study personnel prior to and after treatment in order to ensure compliance and drug accountability. Each MDI will also be weighed at the time of study drug packaging. The weight of each returned MDI will be verified at final reconciliation of return.

Priming of the inhaler is necessary before use. Hence, the number of priming shots will also be recorded and accounted for.

### Adverse Events

### Adverse Event Definition

An adverse event (AE) is any untoward medical occurrence in a patient or clinical investigation subject administered a pharmaceutical product and which does not necessarily have a causal relationship with this treatment.

An AE can therefore be any unfavorable and unintended sign (including an abnormal laboratory finding, for example), symptom or disease temporally associated with the use of the investigational drug, whether or not related to the investigational drug. Any AE will be recorded in the case report form and source documents.

### Severity

The severity of the AE will be characterized as “mild, moderate or severe” according to the following definitions: (1) mild events are usually transient and do not interfere with the subject's daily activities; (2) moderate events introduce a low level of inconvenience or concern to the subject and may interfere with daily activities; and (3) severe events interrupt the subject's usual daily activity.

### Relationship

The causal relationship between the study medication and the AE has to be characterized as unrelated, unlikely, possible or probable. All efforts should be made to classify the AE according to the categories provided below.

Events can be classified as “unrelated” if there is not a reasonable possibility that the study medication caused the AE.

An “unlikely” relationship suggests that only a remote connection exists between the study drug and the reported AE. Other conditions, including chronic illness, progression or expression of the disease state or reaction to concomitant medication, appear to explain the reported AE.

A “possible” relationship suggests that the association of the AE with the study medication is unknown; however, the AE is not reasonably supported by other conditions.

A “probable” relationship suggests that a reasonable temporal sequence of the AE with drug administration exists and, in the investigator's clinical judgment, it is likely that a causal relationship exists between the drug administration and the AE, and other conditions (concurrent illness, progression or expression of disease state or concomitant medication reactions) do not appear to explain the AE.

### Statistical Analysis

### Efficacy Definitions

The efficacy assessments employed in this study are widely used and generally recognized as reliable, accurate and relevant in evaluating response and tolerance to migraine therapy. The following terms will be used in the efficacy analyses:

Pain response is defined as a pain score of 0 (none) or 1 (mild) at a particular time point post-dose without use of rescue medication up to that time point.

Pain free is defined as a pain score of 0 (none) at a particular time point post-dose without use of rescue medication up to that time point.

Pain intensity difference (“PID”) is defined as the difference in pain intensity score at any time point subtracted from the baseline pain intensity score.

Sum of pain intensity differences (“SPID”) is defined as the weighted sum of the pain intensity score differences over different periods from baseline adjusted for the interval between assessments.

Pain relief is defined as the subjective relative pain relief assessed relative to baseline.

Time to onset of meaningful relief is defined as the time interval from baseline to the time of onset of meaningful pain relief as assessed using the stopwatch method.

Relapse (recurrence) is defined as a pain response (none or mild) within the first two hours post-dose, but pain worsened to moderate or severe within the remainder of the 24-hour evaluation period.

Time to relapse is defined as the time interval from two hours post-dose to the time of a pain score of 2 or 3 or the use of rescue medication.

Time to rescue is defined as the time from baseline to the time rescue medication is used.

### Efficacy Variables

The primary measure of efficacy will be the proportion of subjects who experience pain response (pain intensity score of mild or none) at two hours post-dose without use of any rescue medication.

The key secondary efficacy parameters include: (1) proportion with pain response at one hour; (2) proportion pain free at one hour and proportion pain free at two hours; (3) proportion with nausea at one hour and proportion at two hours; (4) proportion with photophobia at one hour and proportion at two hours; (5) proportion with phonophobia at one hour and proportion at two hours; (6) time to onset of meaningful pain relief; (7) pain relief at two hours relative to baseline; (8) SPID at one hour; and (9) proportion using rescue medication within two hours.

Additional secondary efficacy parameters include: (1) proportion with pain response by time point; (2) proportion pain free by time point; (3) proportion with nausea by time point; (4) proportion with photophobia by time point; (5) proportion with phonophobia by time point; (6) SPID at two hours; (7) change in pain intensity score by time point, with emphasis at one and two hours; (8) proportion with relapse; (9) time to relapse; (10) time to use of rescue medication; (11) pain relief by time point; (12) proportion with functional impairment score of 0 or 1 at one hour and proportion at two hours; (13) change in nausea intensity by time point, with emphasis at one hour and at two hours; (14) proportion with vomiting by time point, with emphasis at one hour and at two hours; (15) proportion of subjects with improved PGI score (score of 1 or 2); (16) proportion of subjects with improved PGS score (score of 1 or 2); (17) PGI score (using individual seven points) at each specified time point; and (18) PGS score (using individual five points) at each specified time point.

### Safety

Listings of values for each subject will be presented with abnormal or out of range values for vital signs, clinical laboratory measurements, ECG/continuous telemetry, and physical examination. Descriptive statistics (N, mean, SD, minimum, median, maximum) will be provided for all continuous safety variables. ECG, chest X-ray and physical examination will be summarized in shift tables to show changes from baseline between normal and abnormal findings. Listings will also be provided for concomitant medication use, medical and drug history.

In addition, descriptive statistics (N, mean, SD, minimum, median, maximum) will also be provided for the maximal change from baseline in supine pulse rate and blood pressure at the pre-defined time points from time of dosing to 10 hours post-dose.

Although the invention has been described with respect to specific embodiments and examples, it should be appreciated that other embodiments utilizing the concept of the present invention are possible without departing from the scope of the invention. The present invention is defined by the claimed elements, and any and all modifications, variations, or equivalents that fall within the true spirit and scope of the underlying principles.

1-10. (canceled)

11. A method of treating acute migraine headaches comprising administering to a subject in need thereof an effective amount of dronabinol through a metered dose inhaler obtained from a physician and wherein the method of treating acute migraine headaches further comprises reducing platelet aggregation in the subject.

12. The method of claim 1, wherein the metered dose inhaler comprises about 0.5% delta-9-THC, about 10% dehydrated alcohol, and about 89.5% 1,1,1,2 tetrafluoroethane.

13. The method of claim 1, wherein the metered dose inhaler comprises about 2.0% delta-9-THC, about 10% dehydrated alcohol, and about 88.0% 1,1,1,2 tetrafluoroethane.
